(19) United States
(12) Patent Application Publication
(10) Pub. No.: US 2014/0039801 A1
(43) Pub. Date: Feb. 6, 2014

(54) QUANTISED BIOMETRIC METHOD, APPARATUS AND COMPUTER PROGRAMME

(71) Applicant: Andrew Sapeluk, Dundee (GB)

(72) Inventor: Andrew Sapeluk, Dundee (GB)

(73) Assignee: University of Abertay

(21) Appl. No.: 13/635,526

(22) PCT Filed: Mar. 17, 2011
(86) PCT No.: PCT/GB2011/050530

§ 371 Date: Aug. 6, 2013

(30) Foreign Application Priority Data
Mar. 18, 2010 (GB) 1004574.8

(51) Int. Cl. G01N 33/48

(52) U.S. Cl. CPC G01N 33/48
USPC 702/19

(57) ABSTRACT

A quantised biometric method comprising the steps of: acquiring a value of a biometric measurement variable from a registrant to generate an acquired registrant variable; identifying a container from a preconfigured number of containers each of preconfigured, substantially non-overlapping, upper and lower boundary values, whose boundary values substantially embrace the value of the acquired registrant variable, to generate a registrant container; adapting the upper and lower boundary values of the registrant container so that the acquired registrant variable is centred therein; adapting the upper and lower boundary values of the remaining containers so that they remain substantially non-overlapping with each other and those of the registrant container, to generate a plurality of registrant imposter containers; storing the boundary values of the registrant container and the registrant imposter containers and an acquired identity of the registrant; acquiring a value of the biometric measurement variable from a user to generate an acquired user variable; acquiring from the user a claim to an identity to generate a claimed identity; retrieving the containers of the registrant with the claimed identity to generate the claimed registrant container and claimed registrant imposter containers; deciding that the user has passed the quantised biometric method in the event the boundary values of the claimed registrant container substantially embrace the acquired user variable.

### TECHNICAL FIELD

The present invention relates to a quantised biometric method, apparatus and computer programme and more particularly, but not exclusively, a quantised biometric method, apparatus and computer programme for processing multiple biometric measurement variables.

### BACKGROUND

Biometrics refers to a process for uniquely recognizing a person (or other biological entity) based upon one or more intrinsic physical or behavioral traits thereof. Referring to FIG. 1, a biometric system typically has two operational modes, namely, verification and identification 10. In a verification operational mode, a biometric system receives, from a user 12, a claim to a particular identity. In testing whether the claim is correct (i.e. that the user is who they claim to be), the biometric system performs a one to one comparison 14 between a captured physical or behavioral trait of the user 12 and a stored template (of the trait) uniquely associated with the claimed identity. In contrast, when operating in an identification mode, the biometric system does not receive a claim to a particular identity (i.e. the user 12 is unknown) and the biometric system must compare 14 the captured physical or behavioral trait of the user 12 against a database 16 of templates from plurality of registrants 18 in an effort to identify the user 12.

The above-mentioned comparison processes 14 involve calculating the distance between the captured physical or behavioral trait of the user 12 and the one or more templates. In the event the distance is less than a predefined threshold (Th), the traits are deemed to have come from the same entity as formed the template (i.e. the user 12 is known to the biometric system). Otherwise, the user 12 is deemed to be unknown to the biometric system (i.e. has failed the identification or verification operational modes) and is, for example, denied access by the biometric system to a controlled resource.

The verification and identification operational modes 10 are contingent upon a previous enrolment (or registration) process 20, wherein measurements are acquired by a sensor (not shown) of a one or more physical or behavioral traits of a registrant 18; and the measurements are processed 22 to produce a set of data (i.e. the template) which form a substantially unique representation of the registrant 18 as compared with the general population (not shown).

The level of False Rejections (FR) and False Acceptances (FA) by a biometric system is determined by the setting of the threshold. A number of threshold setting mechanisms are known, including modelling the general population using a world model or by weighting the coefficients of the model vector (or array). However, whilst these techniques can provide good results, they tend to produce optimisations to local minima which are susceptible to changes in the operating environment (e.g. lighting conditions for face recognition and channel spectral properties for speaker recognition).

Performance optimisation tends to have another disadvantage in that, as a biometric system becomes increasingly highly-tuned, it becomes more susceptible to environmental or operating conditions. In many cases, the major sources of performance variation are the so-called human factors. The handling of human factors (including the Human Computer Interaction), can be viewed as a separate pre-process (i.e. the HF pre-processing step 24) whose output is the presentation of a biometric sample suitable for processing by a biometric system during enrolment 20 or in the verification and/or identification operational modes 10.

If the biometric system is tuned for the primary aim of rejecting of an imposter, a failure in either the HF pre-processing 24 or the comparison step 14 of the verification and/or identification operational modes 10 will produce the correct result. Thus, a failure in the HF pre-processing 24 step does not make a False Acceptance (FA) of an imposter more likely. Accordingly, this condition can be ignored.

However, for a registrant to be recognised by the biometric system, the registrant 18 must successfully pass both the HF pre-processing 24 and the comparison step 14 of the verification and/or identification operational modes 10 processes. Accordingly, the registrant's pass rate is the probability of passing the HF pre-processing 24 (henceforth known for simplicity as P_{HF}) multiplied by the probability of passing the comparison step 14 (henceforth known for simplicity as P_{COMP}). Thus, the False Reject rate is given by FR=(1−P_{HF}P_{COMP})×100.

In many biometric systems the only way of ensuring that the human factors do not cause too many False Rejections is to raise the rejection threshold. However, this results in an increased False Acceptance rate.

### SUMMARY OF THE INVENTION

According to the invention there is provided a quantised biometric method, a multivariate quantised biometric method, a quantised biometric apparatus and computer programme as claimed in the appended Claims.

The use of more than one measured variable to improve the error rate of a biometric system is already known. However, whilst it is acknowledged that this approach might improve the FA or FR of a biometric method, it is not known how to combine the multiple measured variables in a manner which improves both FA and FR. The prior art systems are based on the premise of combining the data from multiple measured variables into a single model for a registrant/user.

The preferred embodiment employs a different approach to using multiple measured variables wherein each of the measured variables is processed separately; and the modelling of each such measured variable is of a highly quantised nature. This approach enables the preferred embodiment to reduce the effects of the sometimes significant errors produced by the so-called human factors of biometric systems and the interaction of people with these systems.

More particularly, the preferred embodiment uses a cohort-based classification system to remove the need to use thresholds in the biometric decision-making process.

The preferred embodiment uses cohort based classification for each of the features where the true person will be identified from the feature. Each feature has a quiescent point with a tolerance for each true person; this may be in vector or matrix form and need not be scalar. For each person use artificially constructed cohorts for each of the feature so that the false rejection rate for that feature is as close to zero as possible. The cohort has the effect of quantising of the feature to reduce the effect of the human factors. By processing multiple biometric features separately and independently in this fashion, the preferred embodiment ensures that whilst some impostors will be able to pass some of the biometric verification processes, the chances of the same impostor passing multiple biometric verification processes are increasingly unlikely as the number of biometric verification processes increases (because the measures are statistically independent or significantly independent).

### BRIEF DESCRIPTION OF THE DRAWINGS

An embodiment of the invention will now be described, by way of example only, with reference to the accompanying drawings in which:

FIG. 1 is a block diagram of a one or more operational phases of a typical prior art biometric system;

FIG. 2 is a graph of a bell curve of a population of height measurements with a tolerance region included therein;

FIG. 3 is a block diagram of a passage comprising a plurality of height sensors;

FIG. 4 is a graph of the bell curve of FIG. 2 with a plurality of decision regions defined by the heights of the height sensors of FIG. 3;

FIG. 5 is a block diagram of a classifier based on the decision regions shown in FIG. 4;

FIG. 6 is a flow chart of a prior art quantised biometric method;

FIG. 7a is a block diagram of a classification bin formed during an enrolment phase of the prior art quantised biometric method of FIG. 6;

FIG. 7b is a block diagram of a plurality of classification bins formed during an enrolment phase of a quantised biometric method of the preferred embodiment;

FIG. 8 is a flow chart of the quantised biometric method of a first aspect of the preferred embodiment; and

FIG. 9 is a schematic of a quantised biometric system of the second aspect of the preferred embodiment.

### DETAILED DESCRIPTION OF THE INVENTION

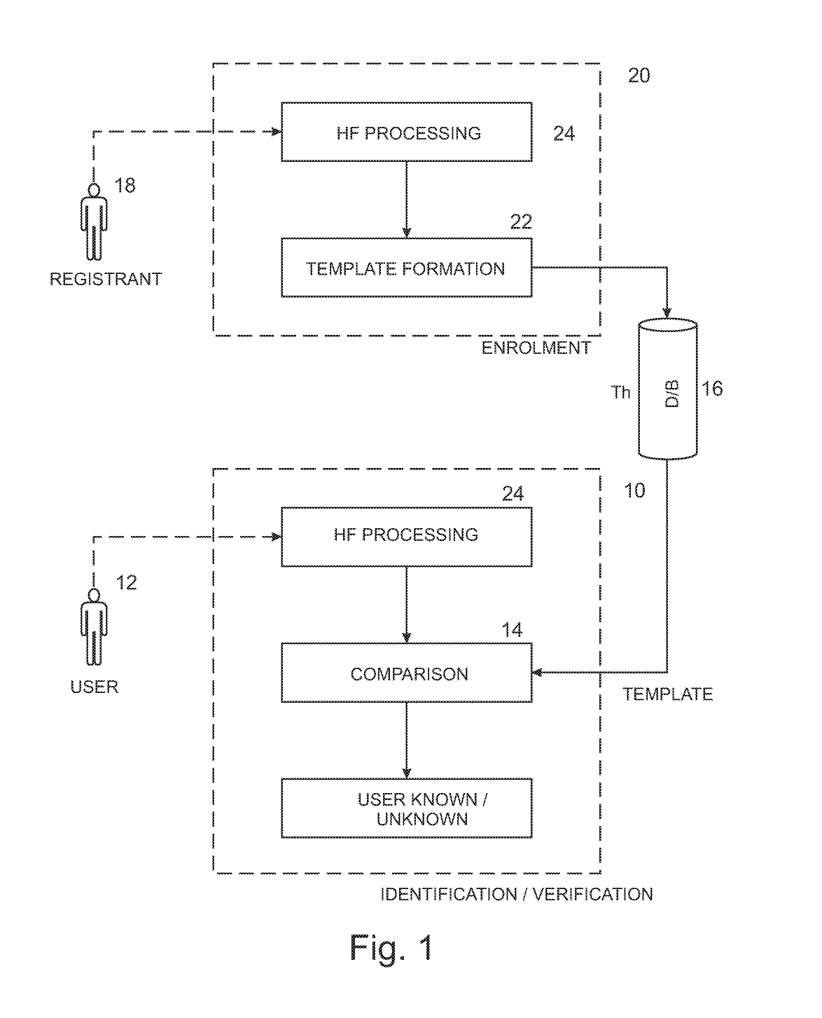

Consider the use of height as a biometric measure. It will be realised that the following description uses a height variable for illustrative purposes only. In particular, the skilled person will realise that the preferred embodiment is not limited to a height measurement variable. On the contrary, the preferred embodiment is applicable to any measurement variable; and particularly (but not exclusively) to non-scalar variables. Indeed, the main benefits of the preferred embodiment are seen when it is applied to multi-dimensional measures and multi-biometrics. Referring to FIG. 1, as will be recalled, the first operational phase of a biometric system is the enrolment phase 20. In the present case, a template of a registrant is formed from a measurement of the height of the registrant. As will be recalled, in the verification operational mode, the biometric system is provided, by a user, with a claim to a particular identity. Thus, in the present case, the biometric system acquires a height measurement of the user and compares it against the height measurement in the template of the registrant whose identity is claimed by the user (for simplicity, this registrant will be referred to henceforth as the claimed registrant). In the event the height of the user matches that of the claimed registrant, the user is deemed to be the claimed registrant. Otherwise, the user is deemed to be an impostor.

Let the height of the registrant be 1.7321564 m (approx 5′9″). Accordingly, a value of 1.7321564 m (or some processed derivative thereof) forms the template for the registrant. It will be understood that the above measurement value is provided for illustrative purposes only. In particular, the skilled person will understand that the preferred embodiment is not limited to a particular value of a height measurement. Instead, as will be seen, the preferred embodiment is capable of capturing and processing substantially any valued height measurement.

In a first example, let the height of the user be 1.7321564 m. In this case, the difference between the height of the registrant and the user is zero. Thus, the user is deemed to be the claimed registrant (i.e. the user has passed the height test of the biometric system). In the event the user's height is 1.7121564 m, the difference between the height of the registrant and the user is 0.02 m. Accordingly, in a biometric system which makes a decision on the basis of exactly matching values of height measurements, the user would be deemed to be an impostor (i.e. the user has failed the height test of the biometric system).

However, if the height measurement is not accurate (i.e. not zero error) a user who is genuinely a registrant (henceforth known, for simplicity, as a true person) will be rejected by the biometric system because the user's height will not exactly equal that of the claimed registrant. This could happen, for example, if the user is wearing shoes of different height than they were during enrolment. Similarly, even if zero measurement error was possible, a small but non-zero number of people would have the same height as the claimed registrant. Thus, these other people (and potential imposters to the registrant) would pass the height test of the biometric system.

In practice, the first of the above-mentioned problems is the most serious because there will always be some error in a measurement. Furthermore, under normal circumstances, it would be expected that a biometric system is more likely to be testing a true person than an imposter. Accordingly, to compensate for the first above-mentioned problem, a tolerance is added to the comparator for the measurement. In the present example, if the tolerance is ±0.03, the user's height can now lie between 1.7021564 and 1.7621564 and the user will still pass the height test of the biometric system.

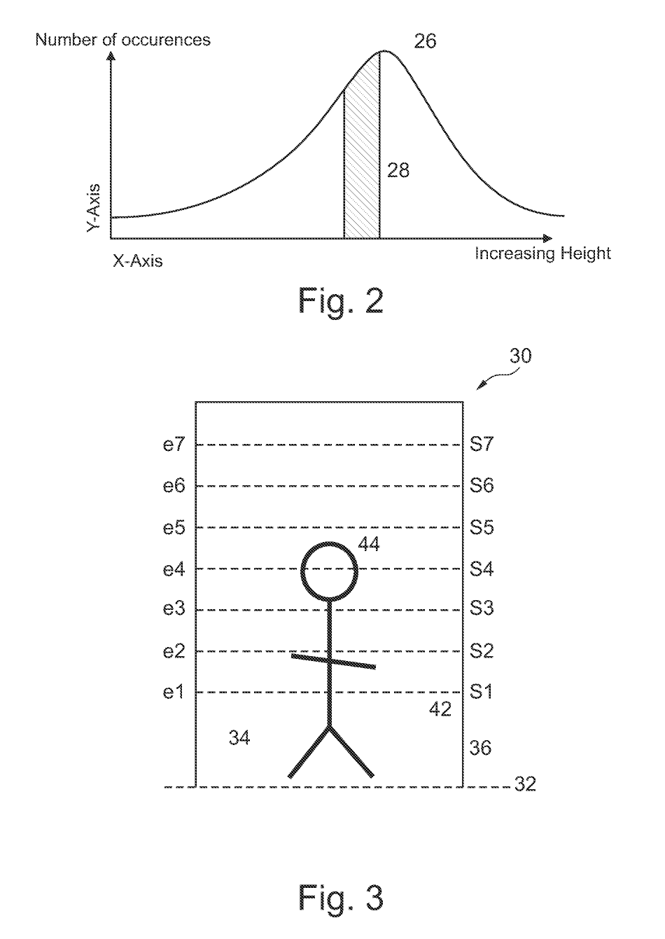

Referring to FIG. 2, the bell curve 26 shows the frequency distribution of the height of the general population, and the base of the shaded region 28 therein defines the acceptable region for passing the height test of the biometric system. In other words, anyone with a height in the shaded region 28 will pass the height test of the biometric system (and be recognised as the registrant). Accordingly, the False Acceptance FA of the biometric system is the area of the shaded region 28 divided by the area under the whole bell curve 26. Thus, widening the shaded region 28 (i.e. increasing the tolerance on the comparator) will decrease the risk of incorrectly rejecting a true person but will increase the risk of incorrectly accepting impostors.

The measurement accuracy in the present example is approximately 0.06/1.732=3.4%. However, this level of accuracy is only achievable with the user's cooperation. In particular, if the user was to stoop slightly, lean to one side, or bend his/her knee and wobble, the measured height would be inaccurate, insofar as the measured height would be less than the user's actual height. Thus, in the event the user is uncooperative, the False Reject rate increases (i.e. the biometric system will reject the true person more often). It is worth noting that whilst many prior art biometric methods (e.g. iris scanning) are very accurate, they also require very co-operative users. This causes these prior art methods to perform very poorly for casual users.

Referring to FIG. 3, one mechanism for overcoming this problem would be to implement a quantised biometric method, through, for example, passage 30 comprising a base member 32 and first and second opposing sides 34, 36 disposed substantially perpendicular to the base member 32. The first side 34 is provided with a plurality of infra-red emitters e1-e7 disposed at different heights relative to the base member 32 and the second side 36 is provided with a plurality of infra-red sensors s1-s7 disposed at corresponding heights to the infra-red emitters e1-e7.

In use, the infra-red emitters e1-e7 produce a plurality of infra-red beams 42 at different heights relative to the base member 32 and the infra-red sensors s1-s7 detect the respective infra-red beams 42. Depending on his/her height relative to the infra-red emitters e1-e7, a user 44 in the passage 30 will break a one or more of the infra-red beams 42. The resulting profile of infra-red signals detected by the infra-red sensors s1-s7 will provide a rough measure of the user's height. More particularly, the highest infra-red beam 42 broken by the user 44 gives a measure of the user's height.

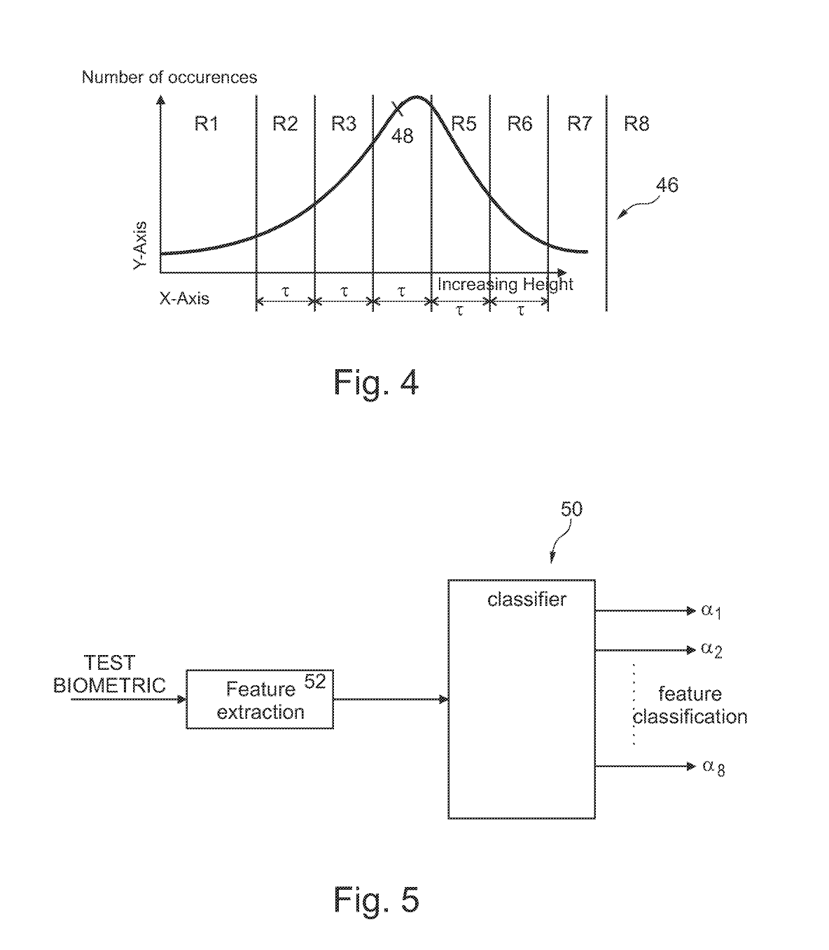

Referring to FIG. 4, the bell curve 46 of the height distribution of the general population is divided into a plurality of regions R1-R8. The centre of each region R1-R8 is defined by the height of the corresponding infra-red emitter e1-e7 in FIG. 3. The width of each such region R1-R8 is defined by the above-mentioned tolerance τ of the comparator (on the height measurement). It will be understood that to ensure the regions R1-R8 are contiguous, the infra-red emitters/sensors are spaced apart in the passage of FIG. 3, by a distance equal to the above-mentioned tolerance. During enrolment in the quantised biometric method, a registrant's height measurement will fall within one of the regions R1-R8. For simplicity, the region in question will henceforth be known as the registrant model.

To verify the identity of a user in the quantised biometric method, the user's height measurement 48 is compared with each of the regions R1-R8 in turn, to determine the region in which the user's height measurement 44 falls. For simplicity, the region in question will be referred to henceforth as the user's height region. In the event the user's height region corresponds to the registrant model of the claimed registrant, the user will be deemed to be the claimed registrant (i.e. the user has passed the height test of the quantised biometric method).

Referring to FIG. 5, this process can be represented as a feature classifier 50, in which any particular feature measurement 52 is classified into one of eight possibilities (also known as classification bins) a1-a8. The classification bins correspond with the regions R1-R8 shown in FIG. 4.

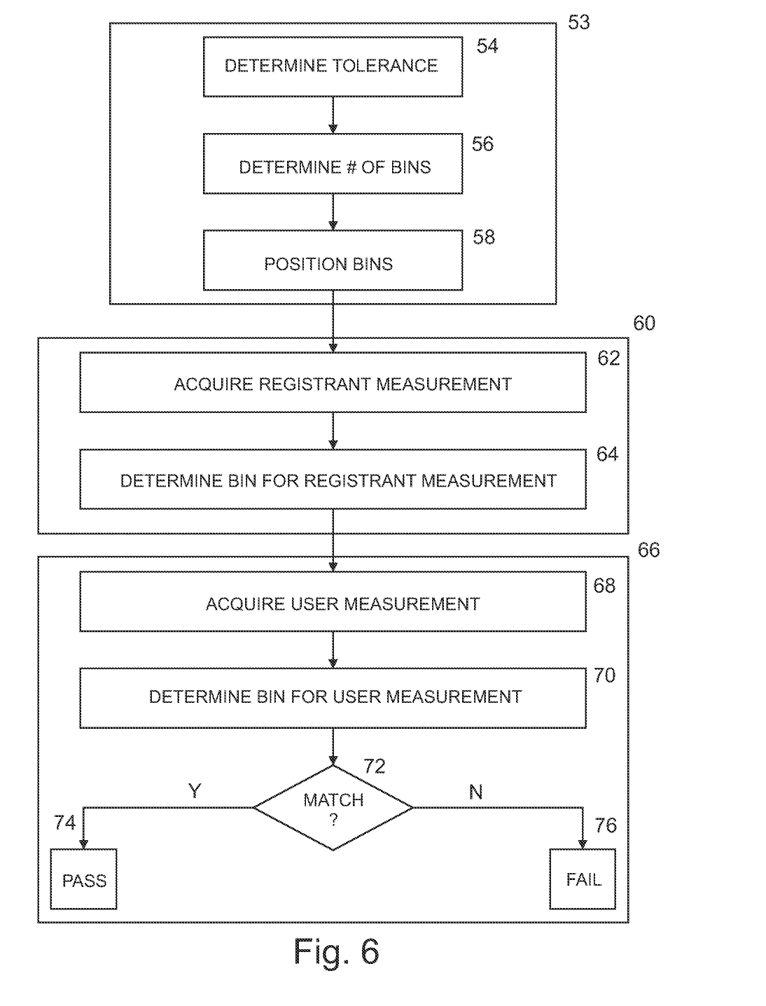

Referring to FIG. 6, and extending from the operational phases shown in FIG. 1 of a prior art biometric method, the quantised biometric method comprises at least three phases, namely a configuration phase 53, an enrolment phase 60 and a identity verification phase 66. The preconfiguration phase 53 comprises the steps of:
defining 54 a tolerance for the comparator;
defining 56 the number of classification bins for the classifier (of FIG. 5); and
determining 58 the relative positioning of the classification bins within the overall measurement range of the relevant measurement variable.

The enrolment phase 60 comprises the steps of:
acquiring 62 a value of the relevant measurement variable of a registrant;
determining 64 the classification bin for the acquired value of the registrant's measurement variable.

The verification phase 66 comprises the steps of:
acquiring 68 a value of the relevant measurement variable of a user;
determining 70 the classification bin for the acquired value of the user's measurement variable;
determining 72 whether the classification bin for the acquired value of the registrant's measurement variable matches the classification bin for the acquired value of the user's measurement variable; and
establishing 74 that the user has passed the quantised biometric method in the event that a match has been found; or
establishing 76 that the user has failed the quantised biometric method in the event that a match has not been found.

The width of the classification bins (i.e. tolerance τ of the comparator) are adapted so that even if a registrant is stooping etc., on a given occasion, the registrant will still be recognised by the quantized biometric system. In other words, the width of the classification bins are tunable to ensure that the false rejection (FR) rate of the quantised biometric system is 0% or as close as practicably possible thereto. In the present example, there are eight different classification bins. Thus, there is a 1 in 8 registrant model of the claimed registrant. Or in other words, the false acceptance (FA) rate of the quantised biometric system is ⅛=12.5%. Thus, the total average error for an equal number of trials=(FA+FR)/2=6.25% and the average accuracy=93.75%.

However, it will be realised that in a quantised biometric system in which the regions (and corresponding classification bins) have fixed positioning determined by the arrangement of the infra-red emitters/sensors in the passage (of FIG. 3), the False Rejection rate will not always be zero. In particular, the templates (i.e. heights in this instance) of some registrants acquired during enrolment will be disposed proximal to the edge of a given classification bin. In view of the above-mentioned sources of measurement error, these registrants are more likely to be falsely classified and hence rejected during the verification operational mode of the quantised biometric system.

The preferred embodiment overcomes this problem by altering the edges of the classification bins on a per registrant basis, so that every registrant's template (i.e. height measurement in this instance) acquired during enrolment, is centered in the classification bin. For brevity, the quantized biometric method of the preferred embodiment will be referred to henceforth as the adaptive quantised biometric method.

Thus, the registrant is more likely to be recognised by the adaptive quantised biometric system during the verification operational mode. In a more general sense, this would amount to training the adaptive quantised biometric system so that the registrant model (or classification bin of the registrant) comprises the registrant's template (i.e. height measurement in this case) with the desired tolerance bounds disposed on either side thereof. The remaining classification bins would be adjusted accordingly. For simplicity and clarity, the remaining classification bins will henceforth be known as imposter models.

In other words, during the enrolment process, the adaptive quantised biometric system creates a registrant model and a plurality of impostor models (i.e. the remaining classification bins) based on the registrant model. The impostor models effectively act as a rejecting mechanism for the adaptive quantised biometric system. In doing so, the adaptive quantised biometric system has removed the need for the threshold operation and replaced it with a simpler operation. More particularly, expanding from the present example of a simple height measurement, to a more complex biometric measurement variable, the acquired values of the measurement variables from the user are compared with all the classification bins, and the one which is closest passes. This is simpler than thresholding.

With a complex multi-dimensional biometric variable, the preferred embodiment allows each dimension to contribute to a decision in a simple way which is not possible using thresholding alone. In particular, in a traditional thresholding approach, dimensional distances are combined in the same way (e.g. Euclidean) prior to thresholding.

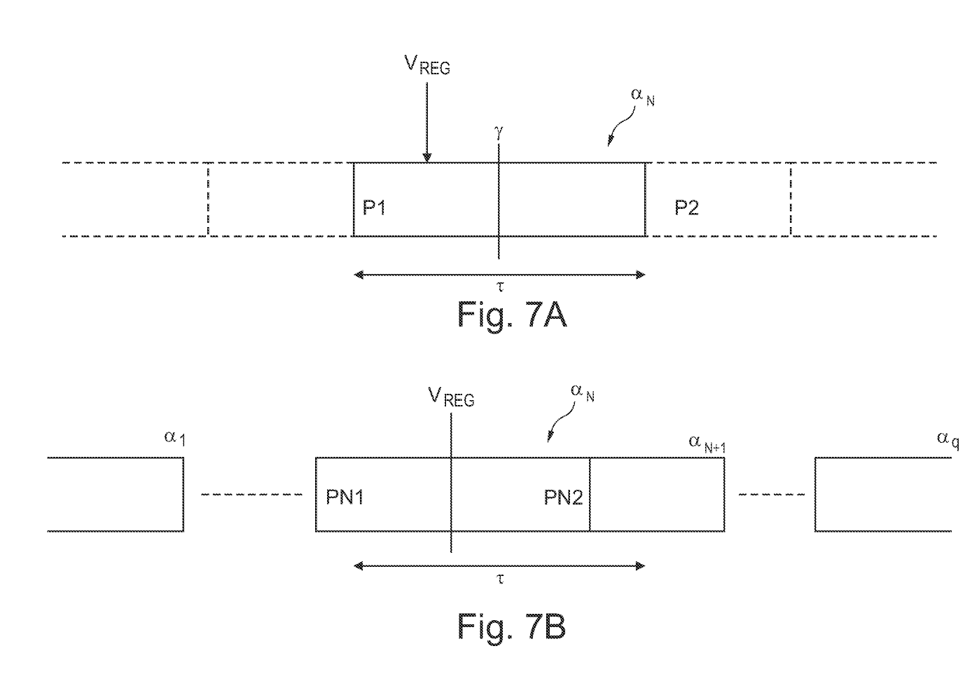

To clarify, referring to FIG. 7a, during the enrolment phase, the prior art quantised biometric method determines the classification bin α_{N} into which the value of a registrant's measurement variable (V_{reg}) falls. The classification bin α_{N} is centred at fixed value γ; and has width τ (defined by the tolerance on the comparator). Depending on the value of the registrant's measurement variable relative to the centre of the classification bin α_{N}; and the tolerance τ on the comparator; the value of a registrant's measurement variable (V_{reg}) may be disposed close to the peripheries (P1, P2) of the classification bin α_{N}.

In contrast, referring to FIG. 7b, during the enrolment phase of the adaptive quantised biometric method, given a set of Q potential classification bins αε [symbol] ^{1×Q}, the adaptive quantised biometric method, forms an initial estimate of the classification bin α_{N} into which the value of a registrant's measurement variable (V_{reg}) falls. Given a predetermined tolerance τ on the comparator, the peripheries (P_{N1}, P_{N2}) of the classification bin α_{N} are adjusted so that the classification bin α_{N} is centred at the value of a registrant's measurement variable (V_{reg}). The peripheries (P_{M1}, P_{M2}, M=1 to N−1) and (P_{O1}, P_{O2}, O=N+1 to Q) of the remaining Q−1 classification bins (α_{M}, M=1 to N−1 and α_{O}, O=N+1 to Q) are adjusted accordingly. It will be realised that the remaining Q−1 classification bins are the above-mentioned imposter models.

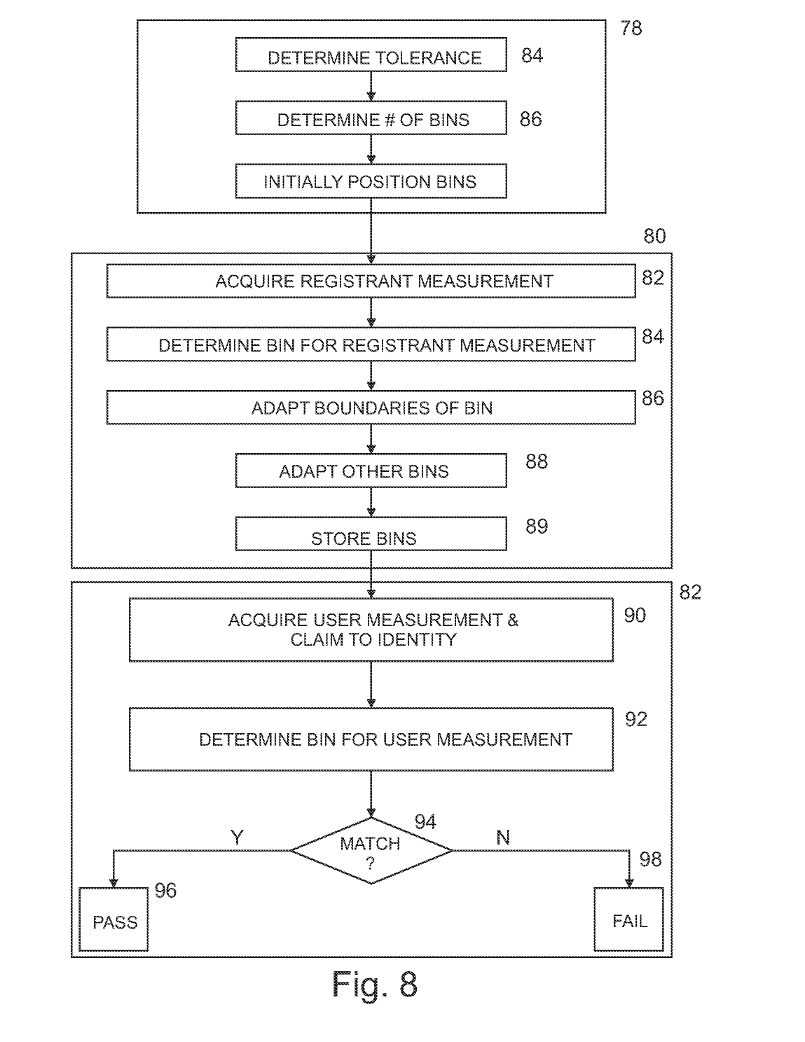

The above discussion has focussed on a single scalar biometric variable. Extending this approach to a one or more vector biometric variables and/or a plurality of scalar biometric variables, and assuming that each of the M elements thereof is represented by the same number Q of classification bins, it can be seen that the set of classification bins is given by αε [symbol] ^{M×Q}. The adaptive quantised biometric method employs the above-mentioned quantisation approach to each of the M elements of the vector and/or scalar biometric variables. More particularly, referring to FIG. 8, the adaptive quantised biometric method comprises at least three phases, namely a configuration phase 78, an enrolment phase 80 and an identity verification phase 82. The preconfiguration phase 78 comprises the steps of:
defining 84 a tolerance for the comparator;
defining 86 the number of classification bins for the classifier; and
determining 88 an initial relative positioning of the classification bins within the overall measurement range of the relevant measurement variable.

The enrolment phase 80 comprises the steps of:
acquiring 82 a value of the relevant measurement variable of a registrant;
determining 84 the classification bin for the acquired value of the registrant's measurement variable (from the initial positioning thereof during the preconfiguration phase 78);
adapting 86 the boundaries of the relevant classification bin, so that the registrant's measurement variable is centered in the relevant classification bin,
adapting 88 the boundaries of the remaining classification bins so that the boundaries of the remaining classification bins abut; and
storing 89 the boundary values of the classification bins for the registrant.

The verification phase 82 comprises the steps of:
acquiring 90 a value of the relevant measurement variable of a user;
receiving 90 a claimed identity from the user;
retrieving the boundary values of the classification bins for the registrant whose identity is claimed by the user;
determining 92 the classification bin whose retrieved boundary values embrace the acquired value of the user's measurement variable;
determining 94 whether the classification bin whose retrieved boundary values embrace the acquired value of the user's measurement variable matches the classification bin for the acquired value of the registrant's measurement variable; and
establishing 96 that the user has passed the quantised biometric method in the event that a match has been found; or
establishing 98 that the user has failed the quantised biometric method in the event that a match has not been found.
   Extending from the present example to a multi-dimensional biometric measurement variable, the classification bins would be applied to each dimension of the variable. Indeed, in the complex biometric and the multi-biometric scenario; for a particular user, the classification bins for different biometric measurement variables will not be in the same order for each variable. In particular, the impostor features will not all fall into the same classification bin relative to the true person but will be varied in distribution.

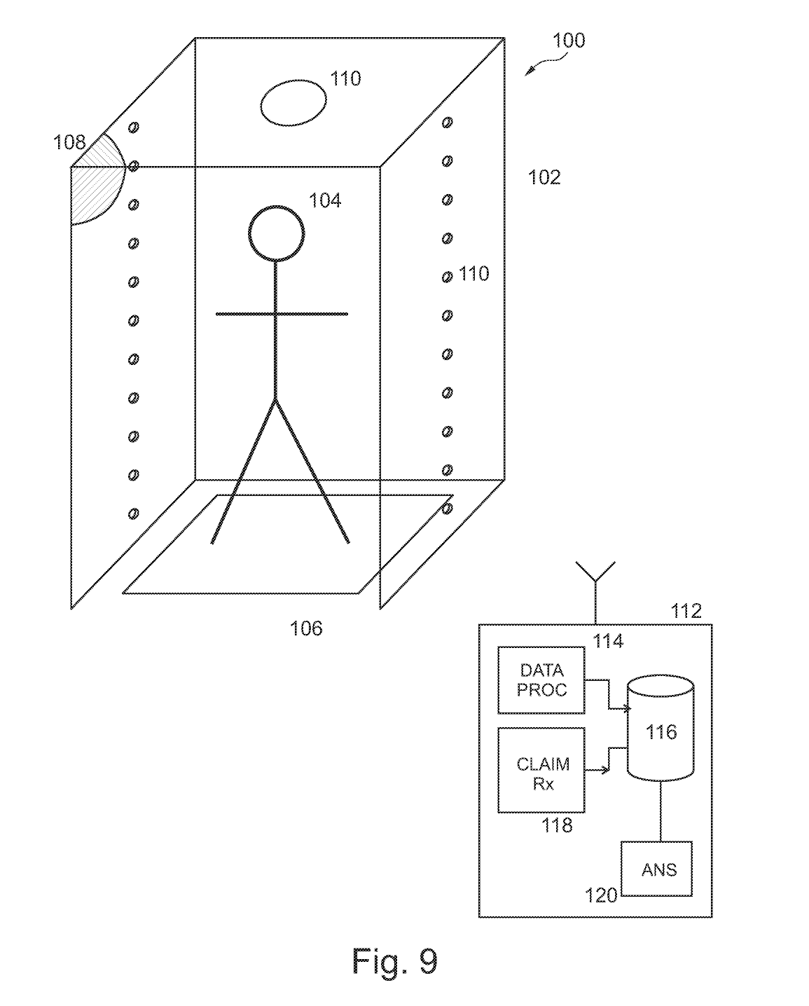

Referring to FIG. 9, a quantised biometric apparatus 100 comprises a booth (in which a user 104 stands) or a tunnel 102 (through which the user 104 walks). The booth/tunnel 102 comprises a weighing scales 106 which measures the weight of the user 104. The booth/tunnel 102 further comprises a camera 108 adapted to film the user in the booth/tunnel 102. The camera 108 is further connectable to a feature recognition facility (not shown) capable of determining for example, the colour of the user's hair or eyes etc. The booth/tunnel 102 further comprises a microphone 110 adapted to record for example the user's voice. The microphone 110 is further connectable to a voice recognition facility (not shown) capable of detecting particular identifying phonic patterns in the user's voice. Similarly, the booth/tunnel 102 comprises a plurality of infra-red or other sensor/emitter couples 110 adapted to detect the user's height. The skilled person will understand that the above-mentioned sensors are provided for illustrative purposes only. In particular, the skilled person will realize that the quantised biometric apparatus 100 is not limited to these sensors, number of sensors or even deployment/arrangement of sensors. Instead, the skilled person will understand that the quantised biometric apparatus 100 is adaptable to include any number of sensors in any given deployment within the booth/tunnel 102.

The sensors from the quantised biometric apparatus 100 are coupleable (wirelessly or hard-wired) to a data processing unit 114 of a biometric processing unit 112. During an enrolment phase, the biometric processing unit 112 determines the registrant models and imposter models for each of the biometric measurement variables of an identified registrant. The registrant and imposter models are stored in a database 116. The biometric processing unit 112 further comprises a claim receiving unit 118. During the verification operational phase, the claim receiving unit 118 receives a claim from the user 104 to a particular identity. The data processing unit 114 separately determines the classification bin for each biometric measurement variable acquired from the user 104. A decision unit 120 compares the classification bin for a given biometric measurement variable against the registrant model for the biometric measurement variable associated with the claimed identity. In the event, of a match, the user is deemed to have passed the adaptive quantised biometric method.

Alterations and modifications may be made to the above without departing from the scope of the invention.

1. A quantised biometric method comprising the steps of:
Acquiring a value of a biometric measurement variable from a registrant to generate an acquired registrant variable;
identifying a container from a preconfigured number of containers each of preconfigured, substantially non-overlapping, upper and lower boundary values, whose boundary values substantially embrace the value of the acquired registrant variable, to generate a registrant container;
adapting the upper and lower boundary values of the registrant container so that the acquired registrant variable is centred therein;
adapting the upper and lower boundary values of the remaining containers so that they remain substantially non-overlapping with each other and those of the registrant container, to generate a plurality of registrant imposter containers;
storing the boundary values of the registrant container and the registrant imposter containers and an acquired identity of the registrant;
acquiring a value of the biometric measurement variable from a user to generate an acquired user variable;
acquiring from the user a claim to an identity to generate a claimed identity;
retrieving the containers of the registrant with the claimed identity to generate the claimed registrant container and claimed registrant imposter containers;
deciding that the user has passed the quantised biometric method in the event the boundary values of the claimed registrant container substantially embrace the acquired user variable.

2. The quantised biometric method as claimed in claim 1 wherein the step of identifying a container from a preconfigured number of containers each of preconfigured, substantially non-overlapping, upper and lower boundary values, whose boundary values substantially embrace the value of the acquired registrant variable, to generate a registrant container; comprises the steps of:
determining a tolerance for the biometric method
determining the upper and lower boundary values according to a desired range of the biometric measurement variable to be spanned by the containers, the tolerance and the number of containers.

3. The quantised biometric method as claimed in claim 1 wherein the method comprises an additional final step of deciding that the user has failed the quantised biometric method in the event the boundary values of the claimed registrant container do not substantially embrace the acquired user variable.

4. A multivariate quantised biometric method comprising the steps of
measuring a plurality of measurement variables from a plurality of registrants;
measuring a plurality of corresponding measurement variables from a user
applying the quantised biometric method as claimed in claim 1 separately to each of the measurement variables;
determining that the user has passed the multivariate quantised biometric method in the event the user has passed at least a predetermined fraction of the applied quantised biometric methods; and
determining otherwise that the user has failed the multivariate quantised biometric method.

5. A quantised biometric apparatus comprising:
a receptacle adapted to receive a user, wherein the receptacle comprises a one or more sensors adapted to measure a value of a one or more measurement variables from the user;
a receiver adapted to receive the or each value of the or each measurement variable from the sensors;
a plurality of contiguous containers, at least some of which are adapted to receive a designated one of the measurement variables;
an identifier adapted to identify the container whose upper and lower boundary values embrace the value of the designated measurement variable;
a first boundary value adjuster adapted to adjust the upper and lower boundaries of the identified container, so that the designated measurement variable is centred therein;
a second boundary value adjuster adapted to adjust the upper and lower boundaries of the remaining containers so that they do not overlap with each other or those of the identified containers;
a claim receiver adapted to receive a verification request from a secondary user, the verification request comprising an identity marker of a registered user of the quantised biometric apparatus;
a data processor adapted to determine whether a measured value of a measurement variable of the secondary user is embraced by the upper and lower boundaries of a container that matches the container for the corresponding measurement variable of the registered user; and
a decision unit adapted to decide that the secondary user has passed the quantised biometric apparatus in the event a match is found.

6. The quantised biometric apparatus as claimed in claim 5 wherein the sensors are adapted to measure a value of a one or more physiological variables of the user.

7. The quantised biometric apparatus as claimed in claim 5 wherein the sensors are adapted to measure a value of a one or more behavioural variables of the user.

8. The quantised biometric apparatus as claimed in claim 5 wherein the sensors are wirelessly coupleable to the receiver.

9. The quantised biometric apparatus as claimed in claim 5 wherein the data processor and decision unit are located remotely from the receptacle.

10. The quantised biometric apparatus as claimed in claim 5 wherein the data processor and decision unit are coupleable to an archive of stored containers and identity markers of registered users of the quantised biometric apparatus.

11. A quantised biometric security computer program, tangibly embodied on a computer readable medium, the computer program product including instructions for causing a computer to execute the quantised biometric method as claimed in claim 1.
